(19) United States
(12) Patent Application Publication
(10) Pub. No.: US 2026/0243725 A1
(43) Pub. Date: Aug. 20, 2026

(54) APPARATUS AND METHOD FOR DETERMINING COULOMBIC EFFICIENCY OF LITHIUM METAL BATTERY

(71) Applicants: Hyundai Motor Company, Seoul (KR); Kia Corporation, Seoul (KR); Pusan National University Industry-University Cooperation Foundation, Busan (KR)

(72) Inventors: Kyungju Nam, Hwaseong (KR); Seunghyo Noh, Hwaseong (KR); Un Hwan Lee, Busan (KR); Joonhee Kang, Busan (KR)

(21) Appl. No.: 19/261,005

(22) Filed: Jul. 7, 2025

(30) Foreign Application Priority Data
Feb. 17, 2025 (KR) 10-2025-0019930

(51) Int. Cl. G01N 27/42; G01N 27/416; H01M 10/052; H01M 10/0561; H01M 10/48

(52) U.S. Cl. CPC G01N 27/423; G01N 27/4166; H01M 10/0561; H01M 10/48; H01M 10/052

(57) ABSTRACT

The present disclosure relates to an apparatus and method for determining coulombic efficiency of a lithium metal battery. An apparatus for determining coulombic efficiency of a lithium metal battery according to the present disclosure includes a physical property data generation unit configured to generate physical property data of an electrolyte in the lithium metal battery, and a coulombic efficiency determination unit configured to determine the coulombic efficiency of the lithium metal battery based on the physical property data of the electrolyte, and elemental composition data representing respective amounts of elements present in the electrolyte.

### CROSS-REFERENCE TO RELATED APPLICATION

This present application claims under 35 U.S.C. § 119 (a) the benefit of Korean Patent Application No. 10-2025-0019930, entitled “APPARATUS AND METHOD FOR DETERMINING COULOMB EFFICIENCY OF A LITHIUM METAL BATTERY,” filed on Feb. 17, 2025, in the Korean Intellectual Property Office, the entire disclosure of which is incorporated herein by reference.

### BACKGROUND

### Technical Field

The present disclosure relates to an apparatus and method for determining coulombic efficiency of a lithium metal battery, and more particularly, to an apparatus and method for determining coulombic efficiency of a lithium metal battery based on artificial intelligence.

### Background

The important components of a lithium-ion battery include a positive electrode, a negative electrode, and an electrolyte. Recently, with the expansion of the electric vehicle market, lithium metal batteries have been gaining attention as next-generation batteries. A lithium metal battery is a battery in which the existing graphite negative electrode having a theoretical capacity of 372 mAh/g is replaced with a lithium metal negative electrode having a theoretical capacity of 3861 mAh/g.

In general, research on lithium metal batteries aims to improve the battery capacity, lifetime, and stability by adjusting the electrolyte, or exploring new materials, rather than by improving the negative electrode. Existing commercial lithium-ion batteries use LiPF₆ salt and a mixture of solvents such as ethylene carbonate (EC) and propylene carbonate (PC) in appropriate ratios. However, when such electrolytes are used in lithium metal batteries, non-uniform deposition of lithium ions occurs on the negative electrode during charging, causing lithium to grow in a dendritic structure. This results in a decrease in capacity due to lithium loss and a reduction in battery lifetime due to short circuits.

In existing research on electrolytes for lithium metal batteries, the battery lifetime is verified by conducting laboratory experiments based on the types and concentrations of salts and solvents.

However, this experimental approach is time-consuming because of the vast number of possible ratios of salts and solvents.

Another existing approach for researching electrolytes for the lithium metal batteries includes determining coulombic efficiency based on the ratio of fluorine to oxygen in the salts and solvents, an amount of oxygen in the solvents, and the like.

However, this approach suffers from low accuracy because the values used are based on simple elemental content rather than the physical properties of the electrolyte materials.

### SUMMARY

The present disclosure is directed to providing an apparatus and method for determining coulombic efficiency of a lithium metal battery based on types and concentrations of salts and solvents through artificial intelligence using simulation data.

In addition, the present disclosure is directed to providing an apparatus and method for determining coulombic efficiency of a lithium metal battery based on physical properties of electrolyte materials.

Further, the present disclosure is directed to providing an apparatus and method for determining coulombic efficiency of a lithium metal battery, which may explore factors with a highest impact on battery lifetime.

Some embodiments of the present disclosure provide an apparatus for determining coulombic efficiency of a lithium metal battery, the apparatus including a physical property data generation unit configured to generate physical property data of an electrolyte in the lithium metal battery, and a coulombic efficiency determination unit configured to determine the coulombic efficiency of the lithium metal battery based on the physical property data of the electrolyte, and elemental composition data representing respective amounts of elements present in the electrolyte.

The coulombic efficiency determination unit may be configured to determine a degree of influence of each variable in the physical property data of the electrolyte on the coulombic efficiency of the lithium metal battery using a linear regression method.

The coulombic efficiency determination unit may be configured to determine the coulombic efficiency of the lithium metal battery based on the influence of each variable on the coulombic efficiency, the physical property data of the electrolyte, and the elemental composition data.

The physical property data may include a highest occupied molecular orbital (HOMO) level of the salt, a lowest unoccupied molecular Orbital (LUMO) level of the salt, a net charge of the lithium atom, an adsorption energy of the salt adsorbed on a flat surface of lithium atoms, or an adsorption energy of the salt adsorbed on a dendritic surface of lithium atoms, and the elemental composition data may include an amount of carbon included in the salt, or an amount of oxygen included in the solvent.

The coulombic-efficiency determination unit may further comprise a random-forest regression engine trained with training data and evaluated with test data, the model being configured to output, in addition to a predicted coulombic efficiency, a Mean Squared Error (MSE) that quantifies a difference between predicted values and actual values.

Some embodiments of the present disclosure provide a method for determining coulombic efficiency of a lithium metal battery, the method including generating, by a physical property data generation unit, physical property data of an electrolyte in the lithium metal battery, and determining, by a coulombic efficiency determination unit, the coulombic efficiency of the lithium metal battery based on the physical property data of the electrolyte, and elemental composition data representing respective amounts of elements present in the electrolyte.

The determining the coulombic efficiency may include determining a degree of influence of each variable in the physical property data of the electrolyte on the coulombic efficiency of the lithium metal battery using a linear regression method.

The determining the coulombic efficiency may include determining the coulombic efficiency of the lithium metal battery based on the influence of each variable on the coulombic efficiency, the physical property data of the electrolyte, and the elemental composition data.

The physical property data may include an HOMO level of the salt, a LUMO level of the salt, a net charge of the lithium atom, an adsorption energy of the salt adsorbed on the flat surface of lithium atoms, or an adsorption energy of the salt adsorbed on a dendritic surface of lithium atoms, and the elemental composition may include an amount of carbon included in the salt, or an amount of oxygen included in the solvent.

The method may further include, before determining the coulombic efficiency, training a random forest model with training data comprising physical property data, elemental composition data, and experimentally measured coulombic efficiencies, and wherein the determining step comprises inputting the physical property data and elemental composition data of the lithium metal battery into the trained random forest model to obtain (i) the coulombic-efficiency prediction and (ii) and MSE value that indicates predictive performance.

According to the present disclosure, by using artificial intelligence based on simulation data, the coulombic efficiency of the lithium metal battery may be determined with reduced time and cost.

In addition, according to the present disclosure, by predicting the coulombic efficiency based on physical properties of electrolyte materials, the coulombic efficiency of the lithium metal battery may be determined with improved accuracy.

Further, according to the present disclosure, by identifying the key factors that significantly affect battery lifetime, an optimal electrolyte composition may be proposed.

As discussed, the method and system suitably include use of a controller or processer.

In another embodiment, vehicles are provided that comprise an apparatus as disclosed herein.

### BRIEF DESCRIPTION OF THE DRAWINGS

The foregoing and other aspects, features, and advantages, as well as the following detailed description of the embodiments, will be better understood when read in conjunction with the accompanying drawings. However, the present disclosure is not intended to be limited to the details shown in the drawings, and various modifications and structural changes may be made therein without departing from the spirit of the present disclosure and within the scope and range of equivalents of the claims. Like reference numbers and designations in the various drawings indicate like elements.

FIG. 1 is a block diagram of a coulombic efficiency determination apparatus of a lithium metal battery according to some embodiments of the present disclosure.

FIG. 2 is a flowchart illustrating a coulombic efficiency determination method of a lithium metal battery according to some embodiments of the present disclosure.

FIG. 3A is a diagram illustrating a flat surface of lithium atoms and a dendritic surface of lithium atoms.

FIG. 3B is a graph illustrating an adsorption energy of each lithium salt adsorbed on a flat surface of lithium atoms or adsorbed on a dendritic surface of lithium atoms.

FIG. 4A illustrates materials previously studied as electrolytes for lithium metal batteries according to some embodiments of the present disclosure.

FIG. 4B is a graph illustrating highest occupied molecular orbital (HOMO) energies and lowest unoccupied molecular orbital (LUMO) energies of the materials shown in FIG. 4A.

FIG. 4C is a graph illustrating the oxidation number derived from the Bader charge values of the lithium atom for each material shown in FIG. 4A.

FIG. 5A is a comparison table of evaluation metrics and scatter plot illustrating the predicted coulomb efficiencies of lithium metal batteries based on training and test data using an extreme gradient boosting (XGBoost) model according to the present disclosure.

FIG. 5B is a comparison table of evaluation metrics and scatter plot illustrating the predicted coulomb efficiencies of lithium metal batteries based on training and test data using a random forest model according to the present disclosure.

FIG. 5C is a comparison table of evaluation metrics and scatter plot illustrating the predicted coulomb efficiencies of lithium metal batteries based on training and test data using a neural network model according to the present disclosure.

FIG. 5D is a comparison table of evaluation metrics and scatter plot illustrating the predicted coulomb efficiencies of lithium metal batteries based on training and test data using a linear regression model according to the present disclosure.

### DETAILED DESCRIPTION

It is understood that the term “vehicle” or “vehicular” or other similar term as used herein is inclusive of motor vehicles in general such as passenger automobiles including sports utility vehicles (SUV), buses, trucks, various commercial vehicles, watercraft including a variety of boats and ships, aircraft, and the like, and includes hybrid vehicles, electric vehicles, plug-in hybrid electric vehicles, hydrogen-powered vehicles and other alternative fuel vehicles (e.g. fuels derived from resources other than petroleum). As referred to herein, a hybrid vehicle is a vehicle that has two or more sources of power, for example both gasoline-powered and electric-powered vehicles.

The terminology used herein is for the purpose of describing particular embodiments only and is not intended to be limiting of the disclosure. As used herein, the singular forms “a,” “an” and “the” are intended to include the plural forms as well, unless the context clearly indicates otherwise. These terms are merely intended to distinguish one component from another component, and the terms do not limit the nature, sequence or order of the constituent components. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Throughout the specification, unless explicitly described to the contrary, the word “comprise” and variations such as “comprises” or “comprising” will be understood to imply the inclusion of stated elements but not the exclusion of any other elements. In addition, the terms “unit”, “-er”, “-or”, and “module” described in the specification mean units for processing at least one function and operation, and can be implemented by hardware components or software components and combinations thereof.

Although exemplary embodiment is described as using a plurality of units to perform the exemplary process, it is understood that the exemplary processes may also be performed by one or plurality of modules. Additionally, it is understood that the term controller/control unit refers to a hardware device that includes a memory and a processor and is specifically programmed to execute the processes described herein. The memory is configured to store the modules and the processor is specifically configured to execute said modules to perform one or more processes which are described further below.

Further, the control logic of the present disclosure may be embodied as non-transitory computer readable media on a computer readable medium containing executable program instructions executed by a processor, controller or the like. Examples of computer readable media include, but are not limited to, ROM, RAM, compact disc (CD)-ROMs, magnetic tapes, floppy disks, flash drives, smart cards and optical data storage devices. The computer readable medium can also be distributed in network coupled computer systems so that the computer readable media is stored and executed in a distributed fashion, e.g., by a telematics server or a Controller Area Network (CAN).

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about”.

The term “coulombic efficiency” herein refers to the ratio of the total charge extracted during discharge to the charge inserted during charge, expressed as a percentage.

The term “Bader charge value” herein refers to the net electronic charge assigned too an atom by a Bader topological partitioning of the charge density.

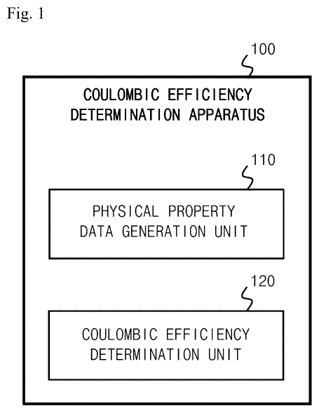

Hereinafter, coulombic efficiency determination apparatus and method of a lithium metal battery according to the present disclosure will be described in detail with reference to FIGS. 1 to 5.

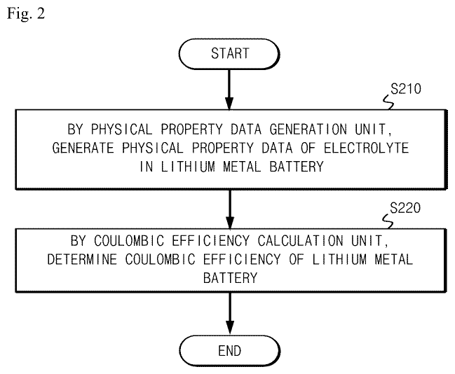

FIG. 1 is a block diagram illustrating a coulombic efficiency determination apparatus 100 of a lithium metal battery according to some embodiments of the present disclosure, and FIG. 2 is a flowchart illustrating a coulombic efficiency determination method of a lithium metal battery according to some embodiments of the present disclosure.

Referring to FIG. 1, a coulombic efficiency determination apparatus 100 of a lithium metal battery according to some embodiments of the present disclosure may include a physical property data generation unit 110, a coulombic efficiency determination unit 120, and the like.

The physical property data generation unit 110 may be configured to generate physical property data of an electrolyte including a lithium salt composed of a lithium cation and an anion, a solvent, and an additive in the lithium metal battery (see S210 of FIG. 2).

For example, the physical property data generation unit 110 may be configured to approximate a highest occupied molecular orbital (HOMO) level of the salt, a lowest unoccupied molecular orbital (LUMO) level of the salt, a net charge of the lithium atom, an adsorption energy of the salt adsorbed on a flat surface of lithium atoms, and an adsorption energy of the salt adsorbed on a dendritic surface of lithium atoms, and to generate the physical property data.

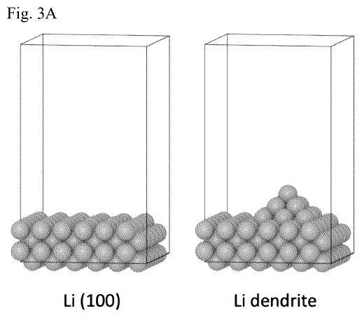

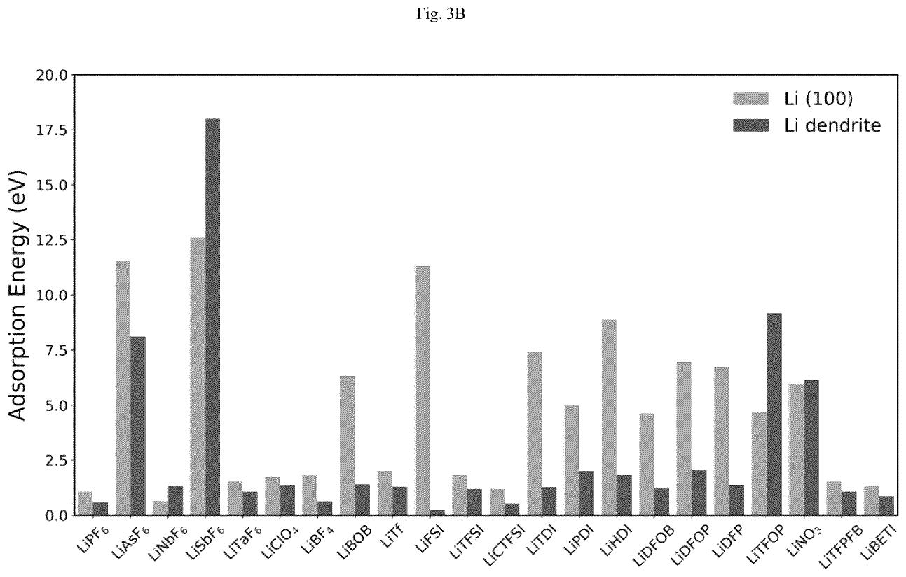

FIG. 3A is a diagram illustrating a flat surface of lithium atoms and a dendritic surface of lithium atoms, and FIG. 3B illustrates, in graph form, an adsorption energy of each lithium salt adsorbed on a flat surface of lithium atoms and when adsorbed on a dendritic surface of lithium atoms. Referring to FIGS. 3A and 3B, an adsorption energy of the salt adsorbed on the flat surface of the lithium atoms may be determined by Equation 1 below.

$${E}_{ad 1} = {E}_{total} - {E}_{100} - {E}_{salt} \qquad [ Equation 1 ]$$

In Equation 1, E_{ad1} is the adsorption energy of the salt adsorbed on the flat surface of the lithium atoms, Eₜₒₜₐₗ is an energy when the salt is attached to the flat surface of the lithium atoms, E₁₀₀ is an energy of the flat surface of the lithium atoms, and Eₛₐₗₜ is an energy of the salt.

Referring to FIGS. 3A and 3B, an adsorption energy of the salt adsorbed on the dendritic surface of the lithium atoms may be determined by Equation 2 below.

$${E}_{ad 2} = {E}_{total} - {E}_{dend} - {E}_{salt} \qquad [ Equation 2 ]$$

In Equation 2, E_{ad2} is the adsorption energy of the salt adsorbed on the dendritic surface of the lithium atoms, Eₜₒₜₐₗ is an energy when the salt is attached to the dendritic surface of the lithium atoms, E_{dend} is an energy of the dendritic surface of the lithium atoms, and Eₛₐₗₜ is the energy of the salt.

The physical property data generation unit 110 may be configured to determine a difference between a highest occupied molecular orbital (HOMO) energy and a lowest unoccupied molecular orbital (LUMO) energy of the lithium salt through orbital analysis using a density functional theory (DFT) calculation.

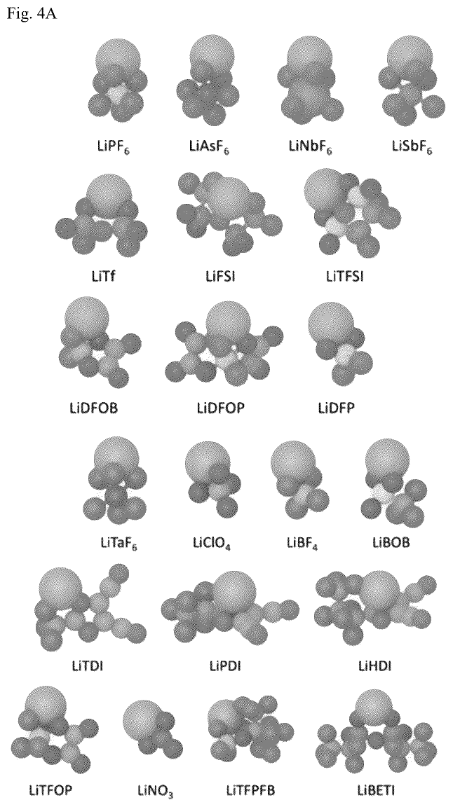

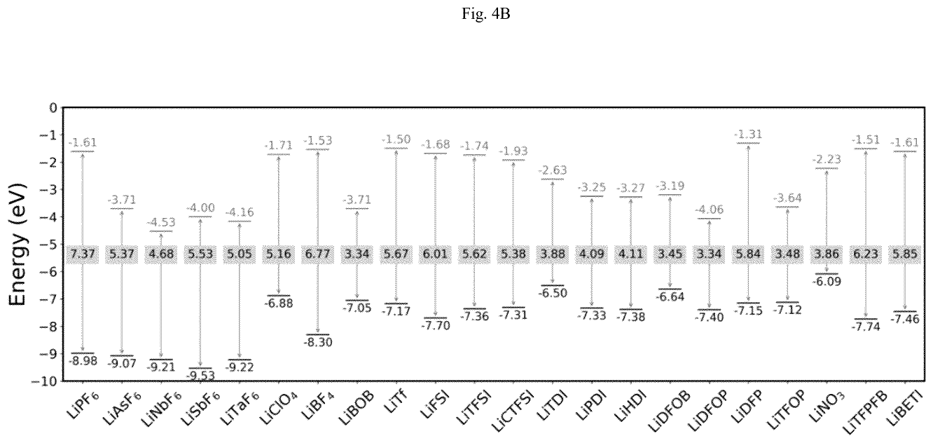

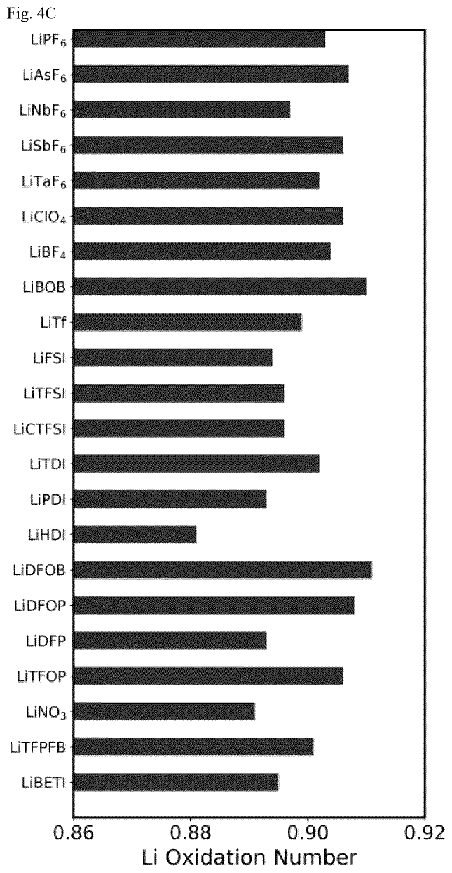

FIG. 4A illustrates stable structures of materials previously studied as electrolytes for lithium metal batteries according to some embodiments of the present disclosure, and FIG. 4B is a graph illustrating highest occupied molecular orbital (HOMO) energies and lowest unoccupied molecular orbital (LUMO) energies based on the stable structures of the materials shown in FIG. 4A.

Referring to FIGS. 4A and 4B, the HOMO refers to the highest orbital occupied by electrons, and the LUMO refers to the lowest orbital unoccupied by electrons. The HOMO level and the LUMO level are indicators related to the oxidation and reduction of a molecule.

FIG. 4C is a graph illustrating the oxidation number derived from the Bader charge values of the lithium atom for each material shown in FIG. 4A.

The physical property data generation unit 110 may be configured to approximate a Bader charge value of the lithium atom through Bader charge analysis using DFT calculations, and to determine an oxidation number of the lithium atom based on the Bader charge value.

For example, the Bader charge value of the lithium atom may be approximated by performing a DFT calculation to obtain the electron density of a lithium salt, determining the charge distribution of each atom in the lithium salt, defining the atomic region associated with the lithium atom, and determining the total charge within that region. The Bader charge value of the lithium atom serves as a computational indicator that reflects the gain or loss of electron density resulting from interactions with surrounding salts.

The coulombic efficiency determination unit 120 may be configured to determine the coulombic efficiency of the lithium metal battery based on the physical property data of the electrolyte, and the amounts of elements defined by a composition of the electrolyte, including an amount of carbon included in the salt, or an amount of oxygen included in the solvent (see S220 of FIG. 2). The coulombic efficiency is an experimentally measured value related to battery lifetime.

For example, the coulombic efficiency determination unit 120 may be configured to determine a degree of influence of each variable in the physical property data of the electrolyte on the coulombic efficiency of the lithium metal battery using a linear regression method.

The physical property data of the electrolyte used in some embodiments of the present disclosure includes DFT-based physical property data that has never been used in existing lifetime prediction methods.

For example, the coulombic efficiency of the lithium metal battery may be determined by Equation 3 below.

$$CE = 0.035 [ FO ] + 0.09 [ InOr ] + 0.102 [ d\_bader ] + 0.028 [ d\_LUMO ] - 3.651 [ sO ] - 2.081 [ aC ] - 0.015 [ d\_ad ] \qquad [ Equation 3 ]$$

In Equation 3, CE is the coulombic efficiency, FO is the fluorine-to-carbon ratio, InOr is the inorganic-to-organic ratio, d_bader is the Bader charge value of the lithium atom, d_LUMO is the LUMO level, sO is the oxygen ratio in the solvent, aC is the anionic carbon ratio, and d_ad is a value obtained by subtracting E_{ad1} from E_{ad2}, where E_{ad1} is the adsorption energy of the salt adsorbed on the flat surface of the lithium atoms, and E_{ad2} is the adsorption energy of the salt adsorbed on the dendritic surface of the lithium atoms.

The relative importance of each variable with respect to the coulombic efficiency may be evaluated based on regression coefficients obtained through multiple linear regression analysis.

For example, the battery efficiency increases when variables such as [FO], [InOr], [d_bader], and [d_LUMO] are retained, and also increases when variables such as [sO], [aC], and [d_ad] are removed.

When the fluorine content is high, stable lithium fluoride may be formed. Lithium tends to lose electrons and thus has a negative Bader charge, but when the lithium interacts too strongly with the salt, the ion conductivity may be reduced.

In addition, previous studies have shown that the battery lifetime increases as the oxygen content in the solvent decreases. A higher LUMO level reduces electrolyte side reactions, thereby extending the battery lifetime. Furthermore, the lifetime also increases when the salt is more strongly adsorbed on the flat lithium surface than on the dendritic surface.

The coulombic efficiency determination unit 120 may be configured to determine the coulombic efficiency of the lithium metal battery based on the influence of each variable on the coulombic efficiency, the physical property data of the electrolyte, and the amounts of elements defined by the composition of the electrolyte.

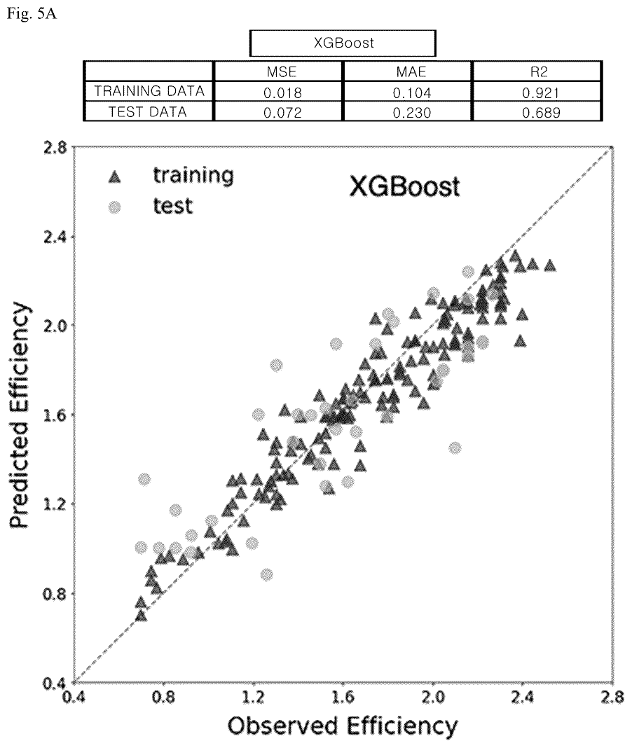

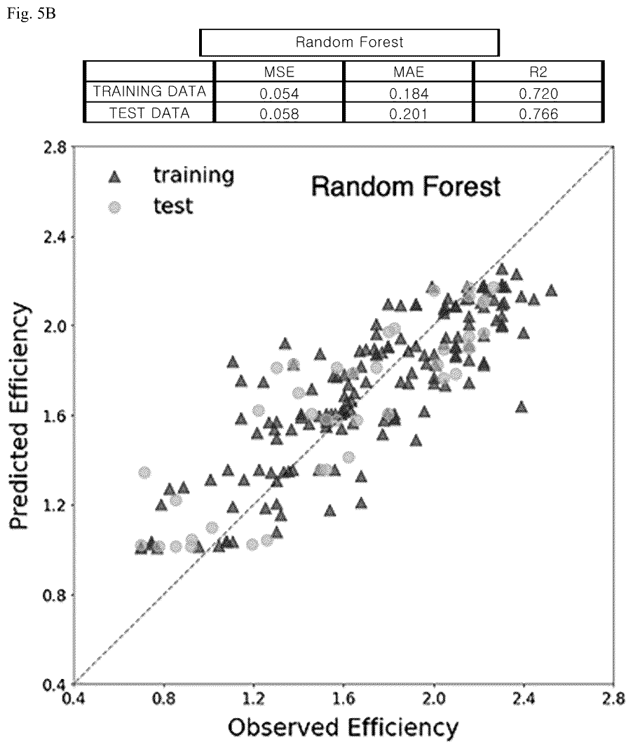

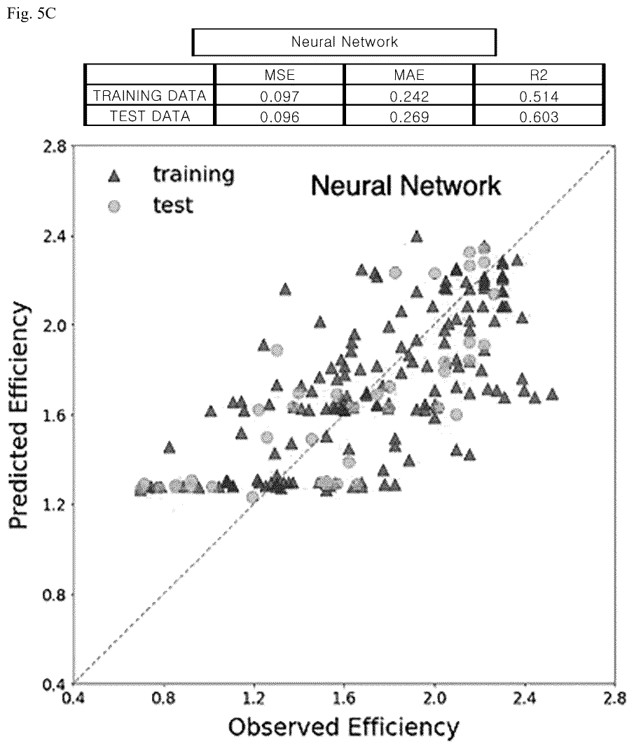

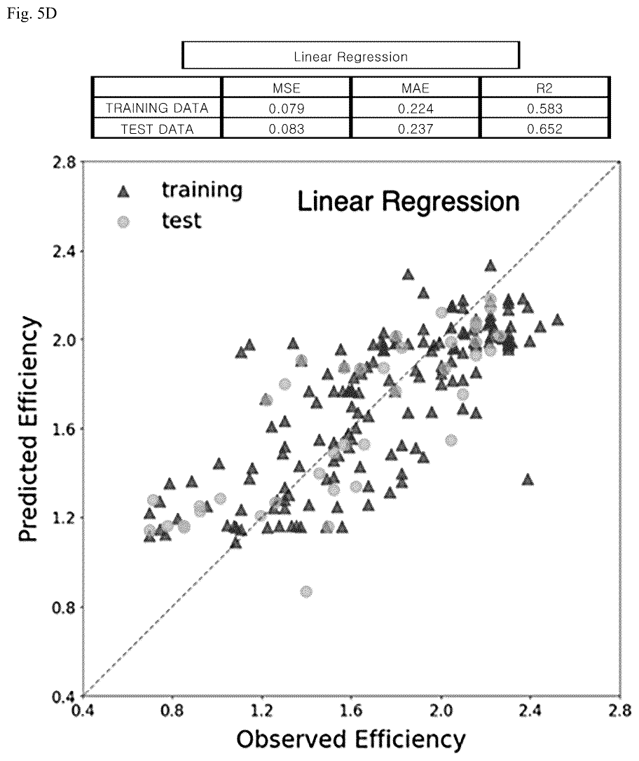

FIG. 5A is a graph illustrating training and predicted data for determining coulomb efficiencies of lithium metal batteries using an XGBoost model according to the present disclosure, and FIG. 5B is a graph illustrating training and predicted data for determining coulomb efficiencies of lithium metal batteries using a random forest model according to the present disclosure.

FIG. 5C is a graph illustrating training and predicted data for determining coulomb efficiencies of lithium metal batteries using a neural network model according to the present disclosure, and FIG. 5D is a graph illustrating training and predicted data for determining coulomb efficiencies of lithium metal batteries using a linear regression model according to the present disclosure.

Referring to FIGS. 5A, 5B, 5C, and 5D, blue triangular data points represent training data, and red circular data points represent test data. The closer the data points are to the diagonal line, the better the prediction performance of the model.

In this context, Mean Squared Error (MSE) is the average squared difference between the predicted values and the actual values. The MSE is characterized by a significant increase in value when outliers occur and is therefore commonly used as a loss function in data analysis. A smaller MSE value may indicate that the model has better predictive performance.

Similarly, Mean Absolute Error (MAE) is the average of the absolute errors between the predicted values and the actual values. A smaller MAE value may indicate that the model has better predictive performance.

Furthermore, R², or coefficient of determination, is an indicator of how well the independent variables explain the dependent variable in a regression model. An R² value closer to 1 may indicate that the model has better predictive performance.

In some embodiments of the present disclosure, the XGBoost model, which exhibited the highest accuracy among four evaluated methodologies, was selected for platform development. However, the coulombic efficiency determination method of the lithium metal battery according to the present disclosure is not limited to the selected methodology.

Referring again to Equation 3, among the HOMO and LUMO levels, the LUMO level is expected to play a more significant role in determining battery lifetime. In addition, a lower oxidation number that reflects the Bader charge value of the lithium atom indicates a weaker bonding tendency between lithium and the salt. As a result, lithium-ion conductivity may improve, which may lead to the formation of a more stable SEI layer on the negative electrode of the lithium metal battery, thereby potentially enhancing battery performance.

Referring again to FIGS. 4B and 4C, LiDFP may be identified as the most promising additive, and LiNO₃, LiHDI, and LiPDI may be expected to contribute to performance improvement in that order.

Given that numerous salt material candidates have not yet been explored, and existing salts may be structurally modified, a more efficient method for identifying optimal salt materials is required.

The coulombic efficiency determination method of the lithium metal battery according to some embodiments of the present disclosure uses a trained model that includes not only chemical structure analysis factors such as FO, InOr, sO, or aC, but also computational chemistry data predicted through DFT calculations, including Bader charge, LUMO, or an adsorption energy for lithium atoms. In addition, multiple linear regression analysis is performed in parallel to evaluate the influence of individual variables within the physical property data.

By determining the coulombic efficiency for each electrolyte combination according to the present disclosure, a large number of salt material candidates may be rapidly screened to identify specific salt materials that may function as optimal additives in lithium metal batteries.

The present disclosure described as above is not limited by the aspects described herein and the accompanying drawings. It should be apparent to those skilled in the art that various substitutions, changes and modifications which are not exemplified herein but are still within the spirit and scope of the present disclosure may be made. Therefore, the scope of the present disclosure is defined not by the detailed description, but by the claims and their equivalents, and all variations within the scope of the claims and their equivalents are to be construed as being included in the present disclosure.

The terms, “and”, “or”, and “and/or” as used herein may include a variety of meanings that also are expected to depend at least in part upon the context in which such terms are used. Typically, “or” if used to associate a list, such as A, B or C, is intended to mean A, B, and C, here used in the inclusive sense, as well as A, B or C, here used in the exclusive sense. Unless otherwise specified, the term “A or B” as used herein encompasses A alone, B alone, or both A and B together.

What is claimed is:

1. An apparatus for determining a coulombic efficiency of a lithium metal battery, the apparatus comprising:
a physical property data generation unit configured to generate physical property data of an electrolyte in the lithium metal battery; and
a coulombic efficiency determination unit configured to determine the coulombic efficiency of the lithium metal battery based on (i) the physical property data of the electrolyte and (ii) elemental composition data representing respective amounts of elements present in the electrolyte.

2. The apparatus according to claim 1, wherein the coulombic efficiency determination unit is further configured to determine a degree of influence of each variable in the physical property data of the electrolyte on the coulombic efficiency of the lithium metal battery using a linear regression method.

3. The apparatus according to claim 2, wherein the coulombic efficiency determination unit is further configured to determine the coulombic efficiency of the lithium metal battery based on the degree of influence of each variable on the coulombic efficiency, the physical property data of the electrolyte, and the elemental composition data.

4. The apparatus according to claim 3, wherein the electrolyte comprises a lithium salt composed of a lithium cation and an anion, a solvent, and an additive, and
the physical property data generation unit is further configured to determine an adsorption energy of the salt adsorbed on a flat surface of lithium atoms based on Equation 1:
$${E}_{ad 1} = {E}_{total} - {E}_{100} - {E}_{salt} \qquad [ Equation 1 ]$$
wherein E_{ad1} is the adsorption energy of the salt adsorbed on the flat surface of the lithium atoms, Eₜₒₜₐₗ is an energy when the salt is attached to the flat surface of the lithium atoms, E₁₀₀ is an energy of the flat surface of the lithium atoms, and Eₛₐₗₜ is an energy of the salt.

5. The apparatus according to claim 4, wherein the physical property data generation unit is further configured to determine an adsorption energy of the salt adsorbed on a dendritic surface of the lithium atoms based on Equation 2:
$${E}_{ad 2} = {E}_{total} - {E}_{dend} - {E}_{salt} \qquad [ Equation 2 ]$$
wherein E_{ad2} is the adsorption energy of the salt adsorbed on the dendritic surface of the lithium atoms, Eₜₒₜₐₗ is an energy when the salt is attached to the dendritic surface of the lithium atoms, E_{dend} is an energy of the dendritic surface of the lithium atoms, and Eₛₐₗₜ is an energy of the salt.

6. The apparatus according to claim 5, wherein the physical property data generation unit is further configured to determine a difference between a highest occupied molecular orbital (HOMO) energy and a lowest unoccupied molecular orbital (LUMO) energy of the lithium salt through orbital analysis using a density functional theory (DFT) calculation.

7. The apparatus according to claim 6, wherein the physical property data generation unit is further configured to approximate a Bader charge value of the lithium atom through Bader charge analysis using the DFT calculation, and to determine an oxidation number of the lithium atom based on the Bader charge value.

8. The apparatus according to claim 7, wherein the physical property data comprise a HOMO level of the salt, a LUMO level of the salt, a net charge of the lithium atom, the adsorption energy of the salt adsorbed on the flat surface of lithium atoms, and the adsorption energy of the salt adsorbed on a dendritic surface of lithium atoms, and
the elemental composition data comprises an amount of carbon included in the salt, and an amount of oxygen included in the solvent.

9. The apparatus according to claim 8, wherein the coulombic efficiency determination unit is further configured to determine the coulombic efficiency of the lithium metal battery using an eXtreme gradient boosting (XGBoost) model, with inputs comprising the degree of influence of each variable on the coulombic efficiency, the physical property data of the electrolyte, and the elemental composition data.

10. The apparatus according to claim 1, wherein the coulombic-efficiency determination unit further comprises a random-forest regression engine trained with training data and evaluated with test data, the model being configured to output, in addition to a predicted coulombic efficiency, a Mean Squared Error (MSE) that quantifies a difference between predicted values and actual values.

11. A method for determining coulombic efficiency of a lithium metal battery, the method comprising:
generating, by a physical property data generation unit, physical property data of an electrolyte in the lithium metal battery; and
determining, by a coulombic efficiency determination unit, the coulombic efficiency of the lithium metal battery based on the physical property data of the electrolyte, and elemental composition data representing respective amounts of elements present in the electrolyte.

12. The method according to claim 11, wherein the determining the coulombic efficiency comprises determining a degree of influence of each variable in the physical property data of the electrolyte on the coulombic efficiency of the lithium metal battery using a linear regression method.

13. The method according to claim 12, wherein the determining the coulombic efficiency comprises determining the coulombic efficiency of the lithium metal battery based on the influence of each variable on the coulombic efficiency, the physical property data of the electrolyte, and the elemental composition data.

14. The method according to claim 13, wherein the electrolyte comprises a lithium salt composed of a lithium cation and an anion, a solvent, and an additive, and
the generating the physical property data comprises determining an adsorption energy of the salt adsorbed on a flat surface of the lithium atoms based on Equation 1:
$${E}_{ad 1} = {E}_{total} - {E}_{100} - {E}_{salt} \qquad [ Equation 1 ]$$
wherein E_{ad1} is the adsorption energy of the salt adsorbed on the flat surface of the lithium atoms, Eₜₒₜₐₗ is an energy when the salt is attached to the flat surface of the lithium atoms, E₁₀₀ is an energy of the flat surface of the lithium atoms, and Eₛₐₗₜ is an energy of the salt.

15. The method according to claim 14, wherein the generating the physical property data comprises determining an adsorption energy of the salt adsorbed on a dendritic surface of the lithium atoms based on Equation 2:
$${E}_{ad 2} = {E}_{total} - {E}_{dend} - {E}_{salt} \qquad [ Equation 2 ]$$
wherein E_{ad2} is the adsorption energy of the salt adsorbed on the dendritic surface of the lithium atoms, Eₜₒₜₐₗ is an energy when the salt is attached to the dendritic surface of the lithium atoms, E_{dend} is an energy of the dendritic surface of the lithium atoms, and Eₛₐₗₜ is the energy of the salt.

16. The method according to claim 15, wherein the generating the physical property data comprises determining a difference between a highest occupied molecular orbital (HOMO) energy and a lowest unoccupied molecular orbital (LUMO) energy of the lithium salt through orbital analysis using a density functional theory (DFT) calculation.

17. The method according to claim 16, wherein the generating the physical property data comprises approximating a Bader charge value of the lithium atom through Bader charge analysis using the DFT calculation, and determining an oxidation number of the lithium atom based on the Bader charge value.

18. The method according to claim 17, wherein the physical property data comprise a HOMO level of the salt, a LUMO level of the salt, a net charge of the lithium atom, the adsorption energy of the salt adsorbed on the flat surface of lithium atoms, and the adsorption energy of the salt adsorbed on a dendritic surface of lithium atoms, and
the elemental composition data comprises an amount of carbon included in the salt, and an amount of oxygen included in the solvent.

19. The method according to claim 18, wherein the determining the coulombic efficiency comprises determining the coulombic efficiency of the lithium metal battery using an extreme gradient boosting (XGBoost) model, with inputs comprising the degree of influence of each variable on the coulombic efficiency, the physical property data of the electrolyte, and the elemental composition data.

20. The method according to claim 17, further comprising, before determining the coulombic efficiency, training a random forest model with training data comprising physical property data, elemental composition data, and experimentally measured coulombic efficiencies, and wherein the determining step comprises inputting the physical property data and elemental composition data of the lithium metal battery into the trained random forest model to obtain (i) the coulombic-efficiency prediction and (ii) and MSE value that indicates predictive performance.
